(19) United States
(12) Patent Application Publication
(10) Pub. No.: US 2023/0270535 A1
(43) Pub. Date: Aug. 31, 2023

(54) INFLATABLE DEVICE WITH OVER-PRESSURIZATION PREVENTION

(71) Applicant: Boston Scientific Scimed, Inc., Maple Grove, MN (US)

(72) Inventor: Brian P. Watschke, Minneapolis, MN (US)

(21) Appl. No.: 18/172,921

(22) Filed: Feb. 22, 2023

Related U.S. Application Data
(60) Provisional application No. 63/268,650, filed on Feb. 28, 2022.

(51) Int. Cl. A61F 2/00

(52) U.S. Cl. CPC A61F 2/004

(57) ABSTRACT

An apparatus, comprising an inflatable member, a pump configured to transfer fluid to the inflatable member, and a housing operatively coupled to the inflatable member and to the pump. The housing defining a cavity. The housing including a base member disposed within the cavity defined by the housing and a sealing member disposed within the cavity defined by the housing. The sealing member being configured to move with respect to the base member.

### CROSS-REFERENCE TO RELATED APPLICATION

This application claims priority to U.S. Provisional Patent Application No. 63/268,650, filed on Feb. 28, 2022, entitled “INFLATABLE DEVICE WITH OVER-PRESSURIZATION PREVENTION”, the disclosure of which is incorporated by reference herein in its entirety.

### TECHNICAL FIELD

This disclosure relates generally to bodily implants, and more specifically to bodily implants including a pump and an over-pressurization feature.

### BACKGROUND

Inflatable implants often include one or more pumps that regulate a flow of fluid between different portions of the implantable device. In some cases, an inflatable implant is used as an artificial urinary sphincter. For example, an inflatable cuff may be implanted such that it surrounds a urethra. The cuff may be inflated to apply pressure to the urethra and prevent urine passage. The cuff may be deflated to allow the patient to void. Problems such as erosion of the cuff through the urethra may occur if the cuff delivers too much pressure to the urethra. Some solutions for this problem have included electronic sensors such as fluid or pressure sensors. However, such solutions have drawbacks. For example, many inflatable implants are formed of an elastomer which is subject to deformation and stretching which may render an electronic sensor of the fluid or pressure not ideal. Accordingly, there is a need for a mechanical means for controlling the pressure of an inflatable implant.

### SUMMARY

In a general aspect, an apparatus, comprising an inflatable member, a pump configured to transfer fluid to the inflatable member, and a housing operatively coupled to the inflatable member and to the pump. The housing defining a cavity. The housing including a base member disposed within the cavity defined by the housing and a sealing member disposed within the cavity defined by the housing. The sealing member being configured to move with respect to the base member.

In some embodiments, the sealing member is configured to form a fluidic seal with an inner surface of the housing.

In some embodiments, the housing includes a biasing member disposed within the cavity defined by the housing, the biasing member being disposed between the base member and the sealing member. In some embodiments, the housing includes a spring member disposed within the cavity defined by the housing, the spring member being disposed between the base member and the sealing member.

In some embodiments, the housing includes a projection extending from an inner surface of the housing. In some embodiments, the housing includes a projection extending from an inner surface of the housing and into the cavity defined by the housing, the projection being configured to engage the sealing member.

In some embodiments, the housing includes an adjustment member, the adjustment member being configured to engage the base member. In some embodiments, the housing includes a screw member, the screw member being configured to engage the base member.

In some embodiments, the housing is operatively coupled to the pump via a tubular member. In some embodiments, the housing is operatively coupled to the inflatable member via a tubular member.

In some embodiments, the inflatable member is configured to be placed near a urethra of a patient. In some embodiments, the inflatable member is an inflatable cuff member.

In some embodiments, the apparatus includes a reservoir, the reservoir being operatively coupled to the housing and being configured to receive fluid. In some embodiments, the apparatus includes a reservoir, the reservoir being operatively coupled to the housing via a tubular member and being configured to receive fluid. In some embodiments, the apparatus includes a reservoir, the reservoir being operatively coupled to the housing and being configured to receive fluid, and the housing includes a projection extending from an inner surface of the housing.

According to another implementation, an apparatus includes an inflatable member; a pump configured to transfer fluid to the inflatable member; and a housing operatively coupled to the inflatable member and to the pump, the housing defining a cavity, the housing including a sealing member disposed within the cavity defined by the housing, the sealing member being configured to move from a first position within the cavity of the housing to a second position within the cavity of the housing, the housing including a biasing member configured to engage the sealing member and bias the sealing member into the first position.

In some embodiments, the housing includes a projection member extending from an inner surface of the housing, the projection member configured to engage the sealing member when the sealing member is in the first position. In some embodiments, the housing includes a base member disposed within the cavity defined by the housing. In some embodiments, the housing includes a base member disposed within the cavity defined by the housing, the biasing member is disposed between the base member and the sealing member. In some embodiments, the inflatable member is an inflatable cuff and is configured to be place near and apply pressure to a urethra of a patient.

### BRIEF DESCRIPTION OF THE DRAWINGS

FIG. 1 is a block diagram of an apparatus according to an aspect.

FIG. 2 is a perspective view of an apparatus according to an aspect.

FIG. 3 is a schematic diagram of the apparatus of FIG. 2 with the sealing member in a first position.

FIG. 4 is a schematic diagram of the apparatus of FIG. 2 with the sealing member in a second position.

FIG. 5 is a perspective view of the housing of the apparatus of FIG. 2.

FIG. 6 is a schematic illustration of a pump or pump assembly of the apparatus of FIG. 2.

FIG. 7 is a schematic diagram of an apparatus according to an aspect.

FIG. 8 is a block diagram of an apparatus according to an aspect.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure.

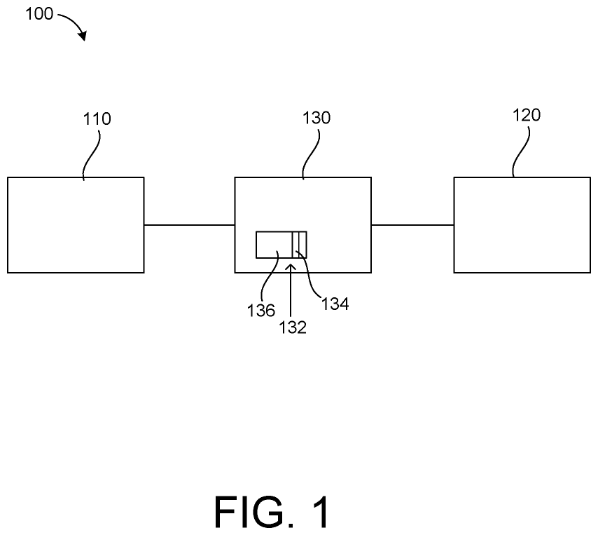

FIG. 1 is a block diagram of an example inflatable implant or device 100. The device 100 includes a pump 110, an inflatable member 120, and a housing 130. The housing 130 is operatively coupled to the pump 110 and to the inflatable member 120. The pump is configured to help transfer fluid to and from the inflatable member. In some embodiments, the pump 110 is an electric pump. In other embodiments, the pump 110 is a manual pump. The device may also include a fluid control system including fluidic components such as one or more valves and the like configured to help transfer fluid to and from the inflatable member 120. In some embodiments, the pump or pump assembly 110 is configured to move fluid between a fluid container and the inflatable member 120. In some embodiments, the fluid container is disposed within or is part of the pump or the pump assembly 110.

The inflatable member 120 is configured to be placed in an inflated configuration and a deflated configuration. Specifically, fluid may be pumped or moved by the pump into the inflatable member 120 to place the inflatable member 120 in the inflated configuration. The fluid may then be removed or at least partially removed from the inflatable member 120 to place the inflatable member 120 in the deflated configuration.

The housing 130 defines a cavity 132 and includes a sealing member 134. The sealing member 134 is configured to form a fluid chamber 136 within the cavity 132 of the housing 130. The sealing member 134 is configured to move within the cavity 132 of the housing 130. Accordingly, as the sealing member 134 moves, a smaller or larger fluid chamber is formed. When the sealing member 134 is positioned such that a larger fluid chamber is formed, a pressure within the inflatable member 120 will be less than when the sealing member 134 is position such that a smaller fluid chamber is formed. In some embodiments, the sealing member 134 may be biased such that when the pressure within the inflatable member 120 is too great (or above a desired amount), the sealing member 134 will be moved against the bias to expand the size of the fluid chamber to lower (or stabilize) the pressure within the inflatable member 120.

In use the inflatable implant or device 100 may be implanted within a body of a patient. In some cases, the inflatable implant or device 100 is an artificial urinary sphincter. In such embodiments, the device 100 may be implanted within the pelvic region of the patient. The inflatable member 120 may be placed such that it provides pressure to the urethra of the patient. In other embodiments, the device is a different type of inflatable implant and is disposed or configured to be disposed within the body of the patient at a different location within the body.

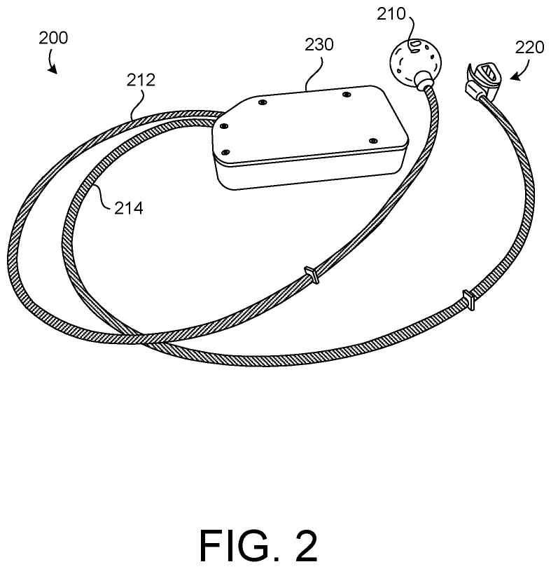

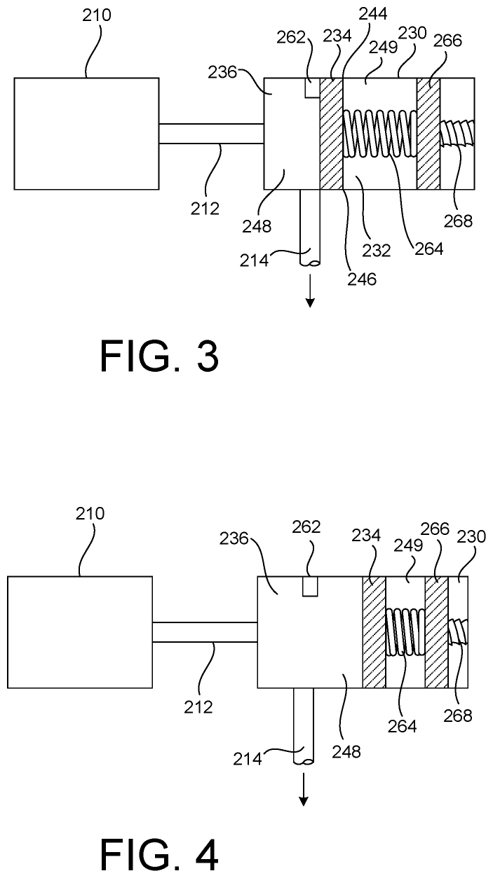

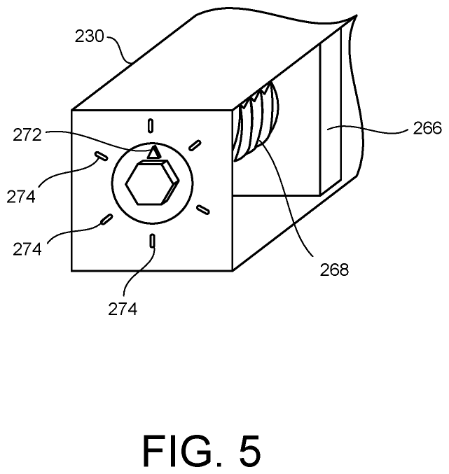

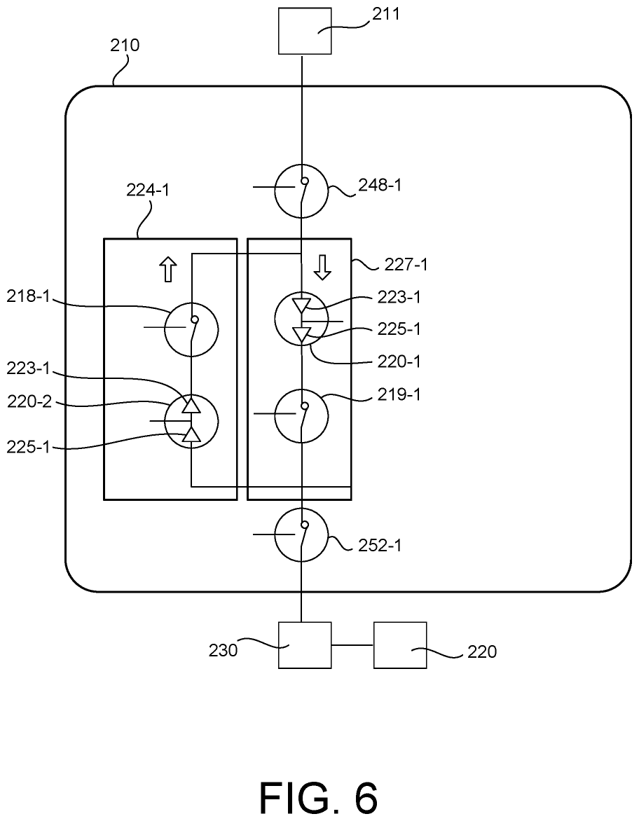

FIGS. 2-6 illustrate an apparatus 200 according to an aspect. FIG. 2 is a perspective view of an apparatus 200. FIG. 3 is a schematic diagram of the apparatus 200 with a sealing member in a first position. FIG. 4 is a schematic diagram of the apparatus 200 with the sealing member in a second position. FIG. 5 is a perspective view of the housing of the apparatus 200. FIG. 6 is a schematic illustration of a pump or pump assembly of the apparatus 200.

The device 200 includes a pump 210, an inflatable member 220, and a housing 230. The housing 230 is operatively coupled to the pump 210 and to the inflatable member 220. In the illustrated embodiment, a tubular member 212 (such as a kink resistant tubing) operatively and fluidically couples the housing 230 to the pump 210. Similarly, a tubular member 214 (such as a kink resistant tubing) operatively and fluidically couples the housing 230 to the inflatable member 220.

In some embodiments, the pump is configured to help transfer fluid to and from the inflatable member. In some embodiments, the pump 210 is an electric pump. In other embodiments, the pump 210 is a manual pump. In some embodiments, the device may also include a fluid control system including fluidic components such as one or more valves and the like configured to help transfer fluid to and from the inflatable member 220.

In some embodiments, as best illustrated in FIG. 6 the device 200 includes a pump or a pump assembly 210. The electronic pump assembly 210 may be an example of the pump or pump assembly 210 of FIG. 1 and/or the electronic pump assembly 210 may include any of the details discussed with reference to the device 100 of FIG. 1.

The electronic pump assembly 210 is configured to transfer fluid to and from the inflatable member 220. In some embodiments, the electronic pump assembly 210 is configured to transfer fluid between a fluid container (such as fluid container 211 in FIG. 6) and the inflatable member. In some embodiments, the fluid container 211 is incorporated into the pump assembly 210. In other embodiments, the fluid container 211 is separate from the pump or the pump assembly 210. The electronic pump assembly 210 may automatically transfer fluid between the fluid container and the inflatable member without the user manually operating a pump (e.g., squeezing and releasing a pump bulb).

The electronic pump assembly 210 includes a pump 220-1 disposed within a fluid passageway 227-1 (e.g., a fill passageway), and an active valve 218-1 disposed within a fluid passageway 224-1 (e.g., an empty passageway). The pump 220-1 may be an electromagnetic pump or a Piezoelectric pump. The pump 220-1 may include a passive check valve 223-1 and a passive check valve 225-1. The fluid passageway 227-1 may be a fluid branch that is separate (and parallel) to the fluid passageway 224-1. The fluid passageway 227-1 is the passageway that transfers fluid from the fluid container to the inflatable member. The fluid passageway 224-1 is the passageway that transfers fluid from the inflatable member to the fluid container. The pump 220-1 is disposed in parallel with the active valve 218-1.

In some examples, the electronic pump assembly 210 may include an active valve 219-1 in series with the pump 220-1 (e.g., the pump 220-1 and the active valve 219-1 are disposed within the fluid passageway 227-1). In some examples, the electronic pump assembly 210 may include a pump 220-2 in series with the active valve 218-1 (e.g., the pump 220-2 and the active valve 218-1 are disposed in the fluid passageway 224-1). The pump 220-2 may be an electromagnetic pump or a Piezoelectric pump. The pump 220-2 may include a passive check valve 223-1 and a passive check valve 225-1. In some examples, the electronic pump assembly 206 includes an active valve 248-1 that is fluidly connected to the fluid container 211. The active valve 248-1 may be in series with either the active valve 218-1 (and the pump 220-2) or the pump 220-1 (and the active valve 219-1). In some examples, the electronic pump assembly 210 includes an active valve 252-1 that is fluidly connected to the inflatable member. The active valve 252-1 may be in series with either the active valve 219-1 (and the pump 220-1) or the pump 220-2 (and the active valve 218-1).

The active valve 248-1, the pump 220-1, the active valve 218-1, the active valve 252-1, the active valve 218-1, and the pump 220-2 may be electronically controlled by a controller and/or driver. The pump 220-1 and the pump 220-2 may be unidirectional or bidirectional. With respect to the fluid passageway 227-1, in some examples, the pump 220-1 and the active valve 219-1 may swap positions (e.g., where the active valve 219-1 is in series between the active valve 248-1 and the pump 220-1). With respect to the fluid passageway 224-1, in some examples, the active valve 218-1 and the pump 220-2 may swap positions (e.g., where the pump 220-2 is in series with and between the active valve 218-1 and the active valve 248-1).

In some examples, one or more additional active valves and/or one or more additional pumps are disposed in series within the fluid passageway 227-1. In some examples, one or more additional active valves and/or one or more additional pumps are disposed in series within the fluid passageway 324-1. In some examples, the electronic pump assembly 210 may include one or more additional (and parallel) fluid passageways, where each additional (and parallel) fluid passageway may include one or more active valves and one or more pumps.

The inflatable member 220 is configured to be placed in an inflated configuration and a deflated configuration. Specifically, fluid may be pumped or moved by the pump 210 into the inflatable member 220 to place the inflatable member 220 in the inflated configuration. The fluid may then be removed or at least partially removed from the inflatable member 220 to place the inflatable member 220 in the deflated configuration. In some embodiments, the pump 210 is configured to move the fluid out of the inflatable member 220 to place the inflatable member 220 in the deflated configuration.

In the illustrated embodiment, the inflatable member 220 is an inflatable cuff (or forms a circle). Accordingly, the device 200 may be used as an artificial sphincter with the inflatable member 220 or cuff placed such that it surrounds or otherwise places pressure on a urethra of a patient. In other embodiments, the inflatable member has a different shape. In some other embodiments, the inflatable member has a different shape and may be place at a different location within the body of the patient. For example, in some embodiments, the inflatable member may have a planar or a tubular shape when place in the inflated configuration.

As best illustrated in FIGS. 3 and 4, the housing 230 defines a cavity 232 and includes a sealing member 234. The sealing member 234 is configured to form a fluid chamber 236 within the cavity 232 of the housing 230. In the illustrated embodiment, the sealing member 234 extends from a first portion 244 of an inner surface 242 of the cavity 232 to a second portion 246 of the inner surface 242 of the cavity 232. The sealing member 234 forms a fluidic seal with the inner surface 242 of the cavity 232. Accordingly, a fluid chamber 248 is formed within the cavity 232. The fluid chamber 248 is configured to receive and house fluid. A second chamber 249 is also formed within the cavity 232 opposite the fluid chamber 248 (the sealing member 234 is disposed between the fluid chamber 248 and the second chamber 249). The fluid is configured to remain in the fluid chamber 248 and outside of the second chamber 249.

The sealing member 234 is configured to move within the cavity 232 of the housing 230. FIG. 3 illustrates the sealing member 234 in a first position. FIG. 4 illustrates the sealing member 234 in a second position. As the sealing member 234 moves, a smaller (as illustrated in FIG. 3) or a larger (as illustrated in FIG. 4) fluid chamber 248 is formed.

When the sealing member 234 is positioned such that a larger fluid chamber 248 is formed (as illustrated in FIG. 4), a pressure within the inflatable member 220 will be less than when the sealing member 234 is position such that a smaller fluid chamber is formed (as illustrated in FIG. 3).

In the illustrated embodiment, the housing 230 includes a stop member 262, biasing member 264, a base member 266, and an adjustment member 268. The biasing member 264 is disposed between and engages the base member 266 and the sealing member 234. The biasing member 264 is configured engage the sealing member 234 to bias or force the sealing member 234 to the first position (such that a smaller fluid chamber 248 is formed) as illustrated in FIG. 3. The stop member 262 is a projection or other member and is configured to engage the sealing member 234 to help retain the sealing member 234 in the first position. In the illustrated embodiment, the stop member 262 extends from the sidewall of the housing 230 and extends into the cavity 232 defined by the housing 230. In other embodiments, the stop member 262 may be a different shape or disposed in a different location.

In the illustrated embodiment, the biasing member 264 is a spring or a spring member. In other embodiments, the biasing member 264 is a different type of biasing member. For example, in some embodiments, the biasing member may be a rod or a piston mechanism.

In some embodiments, the sealing member 234 may be biased such that when the pressure within the inflatable member 220 is too great (or above a desired amount), the sealing member 234 will be moved against the bias to expand the size of the fluid chamber to lower (or stabilize) the pressure within the inflatable member 220. In other words, when the pressure within the inflatable member 220 is too great (or above a desired amount), the sealing member 234 will be moved from the first position (as illustrated in FIG. 3) to the second position (as illustrated in FIG. 4) to expand the size of the fluid chamber to lower (or stabilize) the pressure within the inflatable member 220. The amount of bias against the sealing member 234 may be set such that the sealing member 234 will move when the inflatable member reaches a threshold amount of pressure. For example, the spring constant of the biasing member 264 may be selected such that the sealing member 234 will move when the inflatable member 220 reaches a desired amount of pressure.

In the illustrated embodiment, the base member 266 is configured to move within the cavity 232. Movement of the base member 266 within the cavity may adjust the amount of force that the biasing member 264 places on the sealing member 234. The adjustment member 268 is configured to engage the base member 266 to move the base member 266 within the cavity 232 of the housing 230. In some embodiments, the adjustment member 268 is a screw member such as a set screw. In other embodiments, the adjustment member 268 is a different type of adjustment mechanism. For example, in some embodiments, the adjustment member may be a movable rod or a piston mechanism.

As best illustrated in FIG. 5, the adjustment member 268 may be set (and thereby move the base member 266) as desired from the outside of the housing 230. In the illustrated embodiment, the adjustment member 268 includes an indicator 272. The housing 230 includes markings 274 that indicate a pressure. The adjustment member 268 may be rotated, for example, by physician, to set the location of the base member 266 and thereby set the pressure of the inflatable member 220 at which the sealing member 234 will be moved.

In use the inflatable implant or device 200 may be implanted within a body of a patient. As discussed above, in some cases, the inflatable implant or device 200 is an artificial urinary sphincter. In such embodiments, the device 200 may be implanted within the pelvic region of the patient. The inflatable member 220 may be placed such that it provides pressure to the urethra of the patient. In other embodiments, the device is a different type of inflatable implant and is disposed or configured to be disposed within the body of the patient at a different location within the body.

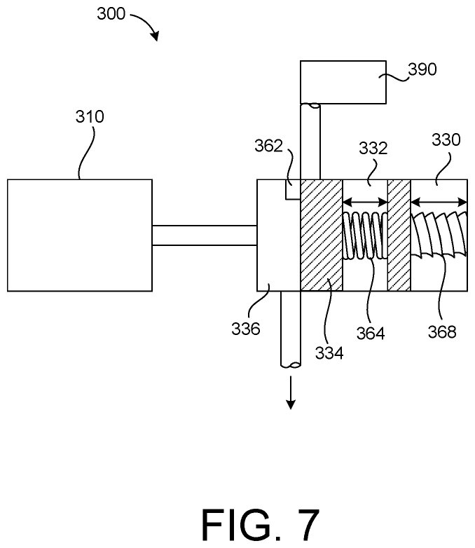

FIG. 7 is a schematic diagram of an apparatus 300 according to an aspect. The device 300 includes a pump 310, an inflatable member 320, a housing 330, and a reservoir 390. The housing 330 is operatively coupled to the pump 310 and to the inflatable member 320. The housing 330 is also operatively coupled to the reservoir 390. The pump is configured to help transfer fluid to and from the inflatable member 320. In some embodiments, the pump 310 is an electric pump. In other embodiments, the pump 310 is a manual pump. The device may also include a fluid control system including fluidic components such as one or more valves and the like configured to help transfer fluid to and from the inflatable member 320.

The inflatable member 320 is configured to be placed in an inflated configuration and a deflated configuration. Specifically, fluid may be pumped or moved by the pump into the inflatable member 320 to place the inflatable member 320 in the inflated configuration. The fluid may then be removed or at least partially removed from the inflatable member 320 to place the inflatable member 320 in the deflated configuration.

The housing 330 defines a cavity 332 and includes a sealing member 334. The sealing member 334 is configured to form a fluid chamber 336 within the cavity 332 of the housing 330. The sealing member 334 is configured to move within the cavity 332 of the housing 330. Accordingly, as the sealing member 334 moves, a smaller or larger fluid chamber is formed. When the sealing member 334 is positioned such that a larger fluid chamber is formed, a pressure within the inflatable member 320 will be less than when the sealing member 334 is position such that a smaller fluid chamber is formed. In some embodiments, the sealing member 334 may be biased such that when the pressure within the inflatable member 320 is too great (or above a desired amount), the sealing member 334 will be moved against the bias to expand the size of the fluid chamber to lower (or stabilize) the pressure within the inflatable member 320. In this embodiment, fluid that enters the fluid chamber 336 when the fluid chamber 336 is in the larger state may flow to the reservoir 390. According, the reservoir 390 is a retainer for fluid that in other embodiments might be in the fluid chamber 336. This embodiment allows for the accommodation of a large amount of fluid to be held without having to move the sealing member 334 a large distance or without having a large enough housing to house such fluid.

In use the inflatable implant or device 300 may be implanted within a body of a patient. In some cases, the inflatable implant or device 300 is an artificial urinary sphincter. In such embodiments, the device 300 may be implanted within the pelvic region of the patient. The inflatable member 320 may be placed such that it provides pressure to the urethra of the patient. In other embodiments, the device is a different type of inflatable implant and is disposed or configured to be disposed within the body of the patient at a different location within the body.

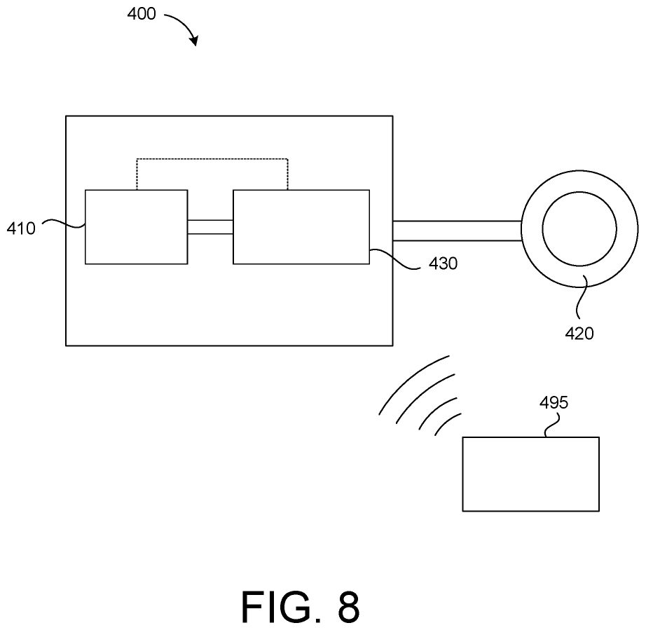

FIG. 8 is a block diagram of an apparatus 400 according to an aspect. The device 400 includes a pump 410, an inflatable member 420, a housing 430, and a controller 495. The housing 430 is operatively coupled to the pump 410 and to the inflatable member 420. The pump 410 is configured to help transfer fluid to and from the inflatable member. In the illustrated embodiment, the pump 410 is an electric pump.

The inflatable member 420 is configured to be placed in an inflated configuration and a deflated configuration. Specifically, fluid may be pumped or moved by the pump into the inflatable member 420 to place the inflatable member 420 in the inflated configuration. The fluid may then be removed or at least partially removed from the inflatable member 420 to place the inflatable member 420 in the deflated configuration.

The housing 430 defines a cavity and includes a sealing member. The sealing member is configured to form a fluid chamber within the cavity of the housing 430. The sealing member is configured to move within the cavity of the housing 430. Accordingly, as the sealing member moves, a smaller or larger fluid chamber is formed. When the sealing member is positioned such that a larger fluid chamber is formed, a pressure within the inflatable member 420 will be less than when the sealing member is position such that a smaller fluid chamber is formed. In some embodiments, the sealing member may be biased such that when the pressure within the inflatable member 420 is too great (or above a desired amount), the sealing member will be moved against the bias to expand the size of the fluid chamber to lower (or stabilize) the pressure within the inflatable member 420.

In the illustrated embodiment, the controller 495 is configured to be operatively coupled to the pump 410 and/or the housing 430 and is configured to control or send signals to operate the device 400. In some embodiments, the controller 495 sends wireless signals to control the device. For example, the controller 495 may be operatively coupled to the pump 410 and/or the housing 430 via Bluetooth, radio frequencies, or another wireless method. In some embodiments, the controller 495 may be operatively coupled to the device 400 while the pump 410 and the housing 430 are disposed within a body of a patient and the controller 495 is disposed outside of the body of the patient.

In some embodiments, the controller 495 is configured to send signals to operate the pump 410. For example, the controller 495 may send signals to instruct or otherwise cause the pump 410 to move fluid into or out of the inflatable member 420. Additionally, in some embodiments, the controller 495 may be configured to send signals to adjust the maximum pressure of the inflatable member 420. For example, the controller 495 may be configured to send signals to move a base member disposed within the housing 430 of the device 400.

In use the inflatable implant or device 100 may be implanted within a body of a patient. In some cases, the inflatable implant or device 100 is an artificial urinary sphincter. In such embodiments, the device 100 may be implanted within the pelvic region of the patient. The inflatable member 120 may be placed such that it provides pressure to the urethra of the patient. In other embodiments, the device is a different type of inflatable implant and is disposed or configured to be disposed within the body of the patient at a different location within the body.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An apparatus, comprising:
an inflatable member;
a pump configured to transfer fluid to the inflatable member; and
a housing operatively coupled to the inflatable member and to the pump, the housing defining a cavity, the housing including a base member disposed within the cavity defined by the housing and a sealing member disposed within the cavity defined by the housing, the sealing member being configured to move with respect to the base member.

2. The apparatus of claim 1, wherein the sealing member is configured to form a fluidic seal with an inner surface of the housing.

3. The apparatus of claim 1, wherein the housing includes a biasing member disposed within the cavity defined by the housing, the biasing member being disposed between the base member and the sealing member.

4. The apparatus of claim 1, wherein the housing includes a spring member disposed within the cavity defined by the housing, the spring member being disposed between the base member and the sealing member.

5. The apparatus of claim 1, wherein the housing includes a projection extending from an inner surface of the housing.

6. The apparatus of claim 1, wherein the housing includes a projection extending from an inner surface of the housing and into the cavity defined by the housing, the projection being configured to engage the sealing member.

7. The apparatus of claim 1, wherein the housing includes an adjustment member, the adjustment member being configured to engage the base member.

8. The apparatus of claim 1, wherein the housing includes a screw member, the screw member being configured to engage the base member.

9. The apparatus of claim 1, wherein the housing is operatively coupled to the pump via a tubular member.

10. The apparatus of claim 1, wherein the housing is operatively coupled to the inflatable member via a tubular member.

11. The apparatus of claim 1, wherein the inflatable member is configured to be placed near a urethra of a patient.

12. The apparatus of claim 1, wherein the inflatable member is an inflatable cuff member.

13. The apparatus of claim 1, further comprising:
a reservoir, the reservoir being operatively coupled to the housing and being configured to receive fluid.

14. The apparatus of claim 1, further comprising:
a reservoir, the reservoir being operatively coupled to the housing via a tubular member and being configured to receive fluid.

15. The apparatus of claim 1, further comprising:
a reservoir, the reservoir being operatively coupled to the housing and being configured to receive fluid,
the housing including a projection extending from an inner surface of the housing.

16. An apparatus, comprising:
an inflatable member;
a pump configured to transfer fluid to the inflatable member; and
a housing operatively coupled to the inflatable member and to the pump, the housing defining a cavity, the housing including a sealing member disposed within the cavity defined by the housing, the sealing member being configured to move from a first position within the cavity of the housing to a second position within the cavity of the housing, the housing including a biasing member configured to engage the sealing member and bias the sealing member into the first position.

17. The apparatus of claim 16, wherein the housing includes a projection member extending from an inner surface of the housing, the projection member configured to engage the sealing member when the sealing member is in the first position.

18. The apparatus of claim 16, wherein the housing includes a base member disposed within the cavity defined by the housing.

19. The apparatus of claim 16, wherein the housing includes a base member disposed within the cavity defined by the housing, the biasing member is disposed between the base member and the sealing member.

20. The apparatus of claim 16, wherein the inflatable member is an inflatable cuff and is configured to be place near and apply pressure to a urethra of a patient.
